(19) United States
(12) Patent Application Publication
(10) Pub. No.: US 2004/0043078 A1
(43) Pub. Date: Mar. 4, 2004

(54) Encapsulation of emulsions

(76) Inventor: David Herault, Bourron-Marlotte (FR)

Correspondence Address: COGNIS CORPORATION, PATENT DEPARTMENT, 300 BROOKSIDE AVENUE, AMBLER, PA, 19002, US

(21) Appl. No.: 10/465,929

(22) PCT Filed: Dec. 18, 2001
(86) PCT No.: PCT/EP01/14920

(30) Foreign Application Priority Data
Dec. 27, 2000 (DE) 100 64 878.9

(51) Int. Cl. A61K 9/16; B01J 13/04; B01J 13/02

(52) U.S. Cl. 424/490; 264/4.1

(57) ABSTRACT

Processes for encapsulating emulsions comprise: (a) providing e a mixture of (i) a polymerizable emulsifier, (ii) at least one polyfunctional comonomer, (iii) at least one hydrophilic component, and (iv) at least one hydrophobic component; and (b) polymerizing the mixture.

### FIELD OF THE INVENTION

This invention relates generally to the encapsulation of emulsions and, more particularly, to encapsulated emulsions, to a process for their production using various polymerizable emulsifiers and comonomers and to their use in surface-active preparations.

### PRIOR ART

“Microcapsules” are understood to be spherical aggregates with a diameter of about 10 nm to about 5 mm which contain at least one solid or liquid core surrounded by at least one continuous membrane. More precisely, they are finely dispersed liquid or solid phases coated with film-forming polymers, in the production of which the polymers are deposited onto the material to be encapsulated after emulsification and coacervation or interfacial polymerization.

The active principles are released from the microcapsules by mechanical, thermal, chemical or enzymatic destruction of the membrane, normally during the use of the preparations containing the microcapsules. Particular disadvantages in this regard are that the microcapsules do not allow controlled release of the active principles from their interior at all or only to an inadequate extent and that the capsules lack stability in the presence of surfactants, especially anionic surfactants. Another disadvantage is that a large quantity of wall or core material is required for encapsulation so that components affording no advantages to the particular application, such as polymers for example, are introduced into the formulation.

The problem addressed by the present invention was to provide a process which would enable emulsions to be encapsulated and the above-described disadvantages to be eliminated.

### DESCRIPTION OF THE INVENTION

The present invention relates to a process for the encapsulation of emulsions, characterized in that a w/o emulsion (i.e. hydrophilic in hydrophobic, for example water in oil) or o/w emulsion (i.e. as o/w=hydrophobic in hydrophilic, for example oil in water) is prepared from (a) a polymerizable emulsifier, (b) at least one polyfunctional comonomer, (c) at least one hydrophilic liquid and (d) at least one hydrophobic liquid, the resulting mixture is polymerized to form a matrix and the hydrophilic and hydrophobic liquids are encapsulated by the matrix.

The present invention relates to encapsulated emulsions obtainable by preparing a w/o or o/w emulsion from

(a) a polymerizable emulsifier,

(b) at least one polyfunctional copolymer,

(c) at least one hydrophilic liquid,

(d) at least one hydrophobic liquid,

(e) optionally monofunctional comonomers and

(f) optionally active substances,

polymerizing the resulting mixture to obtain a matrix and encapsulate the hydrophilic and hydrophobic liquids in the matrix.

It has surprisingly been found that o/w or w/o emulsions containing polymerizable emulsifiers and polyfunctional comonomers can be directly encapsulated in the solution. This is particularly advantageous because it means that no polymers and hence no components unnecessary for the properties of the emulsions have to be added for encapsulation. The invention includes the observation that microcapsules with high wall stability are formed by the use of polymerizable emulsifiers in combination with polyfunctional monomers without any need for large quantities of wall or core material to be used for encapsulation. By virtue of the high stability of the capsules—even in the presence of anionic surfactants—correspondingly encapsulated emulsions are eminently suitable for the stabilization and the controlled release and long-term effect of active substances.

Polymerizable Emulsifier

The polymerizable emulsifiers suitable for the purposes of the invention are made up of a lipophilic part and a hydrophilic part and contain a polymerizable group. This polymerizable group is covalently or ionically bonded to the lipophilic or hydrophilic part or is positioned between the lipophilic and hydrophilic parts. According to the invention, the polymerizable group may be selected from methacryl, aryl, vinyl or allyl groups and is preferably selected from methacryl and acryl groups.

The following are preferred polymerizable emulsifiers:

Nonionic surfactants with OH functionality esterified with acrylic and/or methacrylic acid, for example lauroxy polyethyleneglycol monoacrylate (Blemmer ALE 800, a product of NOF Corporation) Typical examples of nonionic surfactants with OH functionality are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides and glucuronic acid derivatives, fatty acid-N-alkyl glucamides, protein hydrolyzates (especially wheat-based vegetable products), polyol fatty acid esters, sugar esters, sorbitan esters, polysorbates and amine oxides. If the nonionic surfactants contain polylgycolether chains, the latter may have a conventional homolog distribution although they preferably have a narrow homolog distribution.

Soybean oil, linseed oil, sunflower oil, unsaturated fatty alcohols (preferably oleyl or iso-oleyl alcohol), oleic acid mono-, di- and triglycerides and mixtures thereof alkoxylated with 1 to 50 mol ethylene oxide and/or propylene oxide (Weerasooriya Upali, Ester Alkoxylation Technology, J. Surfactants Deterg., (1999), 2(3), 373-381), then epoxidized (Sasaki, Kazuo, Epoxidized Oil, Yuki Gosei Kagaku Kyokai Shi (1975), 33(7), 580 to 5) and then ring-opened with acrylic acid and/or methacrylic acid, such as for example oleic acid diglyceride 20 EO ring-opened with acrylic acid.

Anionic surfactants acidified with an acid, preferably hydrochloric acid, and then neutralized with a basic bifunctional monomer, preferably diallylamine, such as for example dodecyl sulfate acidified with HCl and diallylamine. A basic bifunctional monomer in the present context is understood to be a basic compound containing two polyfunctional groups, i.e. allyl, vinyl, acryl or methacryl groups. Typical examples of anionic surfactants are soaps, alkyl benzene-sulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monolyceride (ether) sulfates, fatty acid amide (ether) sulfates, mono- and dialkyl sulfosuccinates, mono- and dialkyl sulfosuccinamates, sulfotrigly-cerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, the latter may have a conventional homolog distribution although they preferably have a narrow-range homolog distribution.

The polymerizable emulsifiers are used in quantities of 0.1 to 50, preferably 2 to 20 and more particularly 3 to 5 and 12 to 20% by weight, based on the final composition of the emulsion, in the process according to the invention.

Polyfunctional Comonomers

Polyfunctional comonomers suitable for use in the process according to the invention are compounds which contain at least two polymerizable functional groups, such as for example acryl, methacryl, allyl or vinyl groups, such as for example 1,6-hexanediol diacrylates, 1,12-dodecanediol dimethacrylates, dipropylene glycol diacrylates, triethylene glycol dimethacrylates, trimethylolpropane triacrylates, trimethylolpropane ethoxylate triacrylates, glyceryl propoxylate triacrylates, diallylamines, N,N′-diallyltartaric acid diamides, divinylbenzenes and analogous compounds. Preferred polyfunctional comonomers are acrylates and/or methacrylate and, in particular, 1,12-dodecanediol dimethacrylate or triethylene glycol dimethacrylate.

The polyfunctional comonomers are used in quantities of 0.01 to 20, preferably 0.1 to 10 and more particularly 0.5 to 5% by weight, based on the final composition of the emulsion, in the process according to the invention.

Monofunctional Comonomers

Monofunctional comonomers may be used as an optional component (d) in the process according to the invention. Monofunctional comonomers are understood to be compounds which contain a polymerizable functional group, such as for example an acryl, methyacryl, allyl or vinyl group, such as for example acrylic, methacrylic, itaconic, citraconic, maleic, fumaric or vinylbenzoic acid and amides or nitriles thereof; aromatic vinyl compounds, such as for example styrene, methyl styrene, ethyl styrene and chlorostyrene; vinyl compounds, such as for example vinyl chloride and acetate; and vinylidene compounds, such as vinylidene chloride for example. Preferred monofunctional comonomers are acrylic and methacrylic acid and esters and nitriles thereof and vinylidene compounds. These comonomers ensure better polymerization and stabilize the capsules by positive or negative charges.

The polyfunctional comonomers are used in quantities of 0.01 to 20, preferably 0.1 to 10 and more particularly 0.5 to 5% by weight, based on the final composition of the emulsion, in the process according to the invention.

Hydrophilic Liquids

Hydrophilic liquids suitable for use in the process according to the invention are, for example, water, glycerol carbonate, polyols, preferably glycerol or glycols, dimethyl formamide, dimethyl acetamide, dimethyl sulfoxide, N-methylpyrrolidone, glycol ethers, short-chain alcohols (C1 to C8), triacetin or mixtures thereof. Water is preferably used as the hydrophilic liquid.

Polyols suitable for use in accordance with the invention preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Preferred polyols are glycerol, diglycerol, trimethylol propane, pentaerythritol, sorbitol, propylene glycol, butylene glycol, hexylene glycol and polyethylene glycols with an average molecular weight of 100 to 1,000 dalton. Glycerol, diglycerol, trimethylol propane, pentaerythritol, sorbitol and mixtures thereof are particularly preferred.

The hydrophilic liquids are used in quantities of 1 to 99, preferably 5 to 95 and more particularly 10 to 90% by weight, based on the final composition of the emulsion, in the process according to the invention.

Hydrophobic Liquids

Suitable hydrophobic liquids are any aliphatic solvents, aromatic solvents, halogenated aliphatic solvents and halogenated aromatic solvents known to the expert from the prior art, fatty alcohols, oil components, preferably triglycerides, and mixtures thereof.

The following are some examples of suitable hydrophobic liquids:

Higher alcohols (octanols, cyclohexanol), ethers and glycol ethers (diethyl ether, dibutyl ether, anisole, dioxane, tetrahydrofuran, mono-, di-, polyethylene glycol ethers), ketones (acetone, butanone, cyclohexanone), esters (acetic ester, glycol ester), amides and other nitrogen compounds (dimethyl formamide, pyridine, N-methylpyrrolidone, acetonitrile), sulfur compounds (carbon disulfide, sulfolan), nitro compounds (nitrobenzene), halogenated hydrocarbons (dichloromethane, chloroform, tetrachloromethane, tri-, tetrachloroethene, ethylene chloride, chlorofluorocarbons), hydrocarbons (spirits, petroleum ethers, cyclohexane, methyl cyclohexane, decalin, terpene solvents, benzene, toluene, xylenes). In many cases, mixtures of these solvents which combine the solution properties, rather than pure solvents, or solubilizers are used (on economy grounds also).

Fatty alcohols are understood to be primary aliphatic alcohols corresponding to formula (I):

R¹OH (I)

where R¹ is an aliphatic, linear or branched hydrocarbon radical containing 6 to 22 carbon atoms and 0 and/or 1, 2 or 3 double bonds. Typical examples are caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxo synthesis and as monomer fraction in the dimerization of unsaturated fatty alcohols. Preferred fatty alcohols are technical C₁₂₋₁₈ fatty alcohols such as, for example, coconut oil, palm oi, palm kernel oil or tallow fatty alcohol.

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di-and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates such as, for example, Dicaprylyl Carbonate (Cetiol® CC), Guerbet carbonates based on fatty alcohols containing 6 to 18 and preferably 8 to 10 carbon atoms, esters of benzoic acid with linear and/or branched Cr₆₋₂₂ alcohols (for example Finsolv® TN), linear or branched, symmetrical or nonsymmetrical dialkyl ethers containing 6 to 22 carbon atoms per alkyl group such as, for examp,e, Dicaprylyl Ether (Cetiol® OE), ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Paraffins, petroleum ether, terpenes and oil components (preferably isopropyl myristate) are preferably used as the hydrophobic liquids. The hydrophobic liquids are used in quantities of 1 to 99, preferably 5 to 95 and more particularly 10 to 90% by weight, based on the final composition of the emulsion, in the process according to the invention.

Active Substances

In addition, active substances may be added as another component (f) to the process according to the invention. Active substances are understood to include any substances where at least one of the following effects can be of advantage to commercial applications:

controlled release (delayed or sustained), for example of a perfume,

modification of physical properties, for example fine distribution, higher apparent density, flowability,

protection against environmental influences, for example hydrolysis or oxidative degradation,

prevention of any deterioration in flowability, for example of a hygroscopic substance,

reduction of the potential hazard of toxic substances,

modification of taste, odor, color,

masking of chemical reactivity,

separation of incompatible components in formulations.

For example, cosmetic active substances as defined in New Cosmetic Science, T. Mitsui, 1997, Elsevier, p. 148-164, may be used. Preferred active substances are tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, plant extracts and vitamin complexes.

The following compounds may also be used as active substances according to the particular application envisaged:

Substances which are intended to flameproof above all wood and wood materials, plastics and textiles (flame retardants), for example chloroparaffins or hexabromobenzene. Object: reducing the irritating effect of these substances on the skin and mucous membrane, release in the event of fire by opening of the capsule under the effect of heat.

Hardeners or monomers and/or reactive oligomers of two-pack adhesives, for example amines. Two-pack adhesives is the name for chemically setting adhesives where monomers and/or reactive oligomers on the one hand and hardeners on the other hand are present as separate components which are only mixed by the user just before use. Examples of two-pack adhesives are reactive adhesives based on epoxides and aminic hardeners. However, two-pack adhesives are also systems of mixtures of polymerizable components and hardeners which, although inactivated, can be activated, for example, by heat. Examples of such systems are inter alia liquid epoxy resins in which encapsulated amines are dispersed.

Flavors, leavening agents for confectionery, ferments, oils and fats for the food industry. Release by heat, pressure. Effect: for example long-lasting taste, as with chewing gum.

Animal feeds and feed supplements, such as for example vitamins, unsaturated vegetable fats. Effect: protection of these substances against deterioration or oxidative degradation.

Brighteners, bleaches, perfumes or enzymes for household detergents. Effect: solving of incompatibility problems between two components of a laundry detergent (for example enzymes and bleaching agents).

Fertilizers, seeds, insecticides, herbicides, fungicides for the agricultural industry. For example, encapsulation of the insecticide methyl parathion increases persistence and reduces toxicity.

Pharmaceutical active principles. The slow release of a medicament from the microcapsules provides for a sustained effect and prevents overdoses immediately after ingestion.

The active substances are used in quantities of 0 to 50, preferably 0.5 to 45 and more particularly 1 to 5 or 30 to 40% by weight, based on the final composition of the emulsion, in the process according to the invention.

Polymerization

The polymerization takes place between 10 and 100° C., preferably between 15 and 50° C. and more particularly at room temperature by UV irradiation and/or initiators. The decomposition of the initiators can also be initiated by a redox process, for example with lauryl peroxide and FeSO₄. A lipophilic or hydrophilic initiator is preferably used, depending on the system (cf. Comprehensive Polymer Sci. 3, 98-146).

Commercial Applications

The process according to the invention is based on the multifunctionality of the polymerizable components (emulsifier and comonomer) and uses their self-organization at the boundary of the lipophilic/hydrophilic liquid in an emulsion. Accordingly, the process requires only a little wall material to obtain the matrix (core/shell capsules) and the microcapsule dispersion obtained contains no, or hardly any, non-copolymerized or non-encapsulated components. The dispersion may be filtered, depending on particle size and the application envisaged, and may be used as such in surface-active preparations.

One or more polymerizable emulsifiers (0.1 to 50, preferably 2 to 20 and more particularly 3 to 5 and 12 to 20% by weight), one or more polyfunctional comonomers (0.01 to 20, preferably 0.1 to 10 and more particularly 0.5 to 5% by weight), optionally monofunctional comonomers (0.01 to 20, preferably 0.1 to 10 and more particularly 0.5 to 5% by weight), the initiator system (0 to 15, preferably 0.1 to 3% by weight) and optionally the biogenic active substances to be encapsulated (0 to 50, preferably 0.5 to 45 and more particularly 1 to 5 or 30 to 40% by weight) are added to a mixture of 1 to 99, preferably 5 to 95 and more particularly 10 to 90% by weight of hydrophilic liquid and 1 to 99, preferably 5 to 95 and more particularly 10 to 90% by weight of lipophilic liquid and an emulsion is prepared. The polymerizable emulsifiers used stabilize the system long enough for the polymerization process to take place. Alternatively, the initiator system may be subsequently added either completely or in part. After 1 to 24 and preferably after 5 to 10 hours' irradiation and/or heating or simply stirring, depending on the initiator system, the microcapsules produced are used as a suspension or after filtration.

Encapsulated emulsions optionally containing active substances as an additional component (e), droplets or capsules with a particle size of 70 nm to 5 μm, preferably 150 nm to 2 μm and more particularly 300 nm to 0.5 μm are obtained by the process according to the invention. The particle sizes were determined by photoelectron correlation spectroscopy using a Coulter N4 Plus Submicron Particle Sizer.

In addition, the encapsulated emulsions produced by the process may be used in surface-active preparations, such as laundry detergents and dishwashing detergents for example, and in cosmetic and/or pharmaceutical preparations which contain mild surfactants, pearlizing waxes, consistency factors, thickeners, superfatting agents, stabilizers, silicone compounds, fats, waxes, lecithins, phospholipids, antioxidants, deodorizers, antiperspirants, antidandruff agents, swelling agents, insect repellents, self-tanning agents, tyrosine inhibitors (depigmenting agents), hydrotropes, solubilizers, preservatives, perfume oils, dyes and the like as further auxiliaries and additives. Examples of cosmetic and/or pharmaceutical preparations include hair shampoos, hair lotions, foam baths, shower baths, creams, gels, lotions, alcoholic and aqueous/alcoholic solutions, emulsions, wax/fat compounds, stick preparations, powders and ointments.

As described under the heading “active substances”, the encapsulated emulsions may also be used in the fields described there, depending on which compounds are encapsulated (cf. Microencapsulation, Chapter 6: Uses, Ullmann's Encyclopedia of Industrial Chemistry, 5th Edition, Vol. A16, 1990, pages 585-587).

Surfactants

Suitable surfactants are anionic (see above), nonionic, cationic and/or amphoteric surfactants which are normally present in the preparations in quantities of about 1 to 70, preferably 5 to 50 and more particularly 10 to 30% by weight. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Waxes

Suitable waxes are inter alia natural waxes such as, for example, candelilla wax, carnauba wax, Japan wax, espartograss wax, cork wax, guaruma wax, rice germ oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs) and correspond to the following general formula:

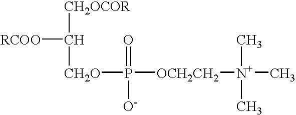

where R typically represents linear aliphatic hydrocarbon radicals containing 15 to 17 carbon atoms and up to 4 cis-double bonds. Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycero-phosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Antioxidants

Antioxidants which interrupt the photochemical reaction chain initiated when UV rays penetrate into the skin may also be added. Typical examples of such antioxidants are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmol/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Self-Tanning Agents and Depigmenting Agents

A suitable self-tanning agent is dihydroxyacetone. Suitable tyrosine inhibitors which prevent the formation of melanin and are used in depigmenting agents are, for example, arbutin, koji acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Preservatives

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Perfume Oils

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Dyes

Suitable dyes are any of the substances suitable and approved for cosmetic purposes as listed, for example, in the publication “Kosmetische Färbemittel” of the Farbstoffkommission der Deutschen Forschungs-gemeinschaft, Verlag Chemie, Weinheim, 1984, pages 81 to 106. These dyes are normally used in concentrations of 0.001 to 0.1% by weight, based on the mixture as a whole.

The total percentage content of auxiliaries and additives may be from 1 to 80% by weight and is preferably from 5 to 50% by weight and more particularly from 7 to 10% by weight, based on the particular composition. The compositions may be produced by standard hot or cold emulsification processes and are preferably produced by the PIT (phase inversion temperature) method.

### EXAMPLES

### Example 1

Blemmer ALE 800 (Lauroxy Polyethylene Glycol Monoacrylate, a Product of NOF Corporation)+Triethyleneglycol Dimethacrylate

97 parts sodium chloride solution (0.01 molar), tetradecane (97), Blemmer ALE 800 (6), triethyleneglycol dimethacrylate (6) and 2,2′-azobis-[2-methyl-N-(2-hydroxyethyl)-propionamide] (0.4) are combined and emulsified under argon for about 1 minute using an Ultraturrax (T₂₅ of IKA Labortechnik, 11,000 to 15,000 rpm). After exposure to UV light (Osram Ultra-Vitalux 300 W) for 1 h, a capsule suspension is obtained.

### Example 2

Blemmer ALE 800+Triethylene Glycol Dimethacrylate

97 parts sodium chloride solution (0.01 M), tetradecane (97), Blemmer ALE 800 (6), triethylene glycol dimethacrylate (6) and lauryl peroxide (0.5) are combined and emulsified under argon with an Ultraturrax (see above). After exposure to UV light (Osram Ultra-Vitalux 300 W) for 1 h, a capsule suspension is obtained.

### Example 3

Blemmer ALE 800+Triethylene Glycol Dimethacrylate

60 parts sodium chloride solution (0.01 M), Blemmer ALE 800 (0.9), triethylene glycol dimethacrylate (0.9) and 2,2′-azobis[2-methyl-N-(2-hydroxyethyl)propionamide] (0.06) are combined. Exposure to UV light (Osram Ultra-Vitalux 300 W) for 2.5 h with stirring produced a slightly cloudy capsule suspension.

### Example 4

Diallyl Ammonium Dodecyl Sulfate+Triethylene Glycol Dimethacrylate

60 parts sodium chloride solution (0.01 M), diallylammonium dodecyl sulfate (0.9), triethyleneglycol dimethacrylate (0.9) and 2,2′-azobis[2-methyl-N-(2-hydroxyethyl)propionamide] (0.06) are combined. Exposure to UV light (Osram Ultra-Vitalux 300 W) for 2.5 h with stirring produced a slightly cloudy capsule suspension (particle diameter as measured by light scattering: 50 to 100 nm).

### Example 5

Blemmer ALE 800+Triethyleneglycol Dimethacrylate

91 parts sodium chloride solution (0.01 M), tetradecane (18), Blemmer ALE 800 (1.5), triethyleneglycol dimethacrylate (1.5), lauryl peroxide (0.1) and FeSO₄ (0.1) are combined and emulsified under argon with an Ultraturrax (see above). After stirring for 18 h and filtration, 17.5 g capsules are obtained as a solid. They release tetradecane on grinding.

### Example 6

Blemmer ALE 800+Triethylene Glycol Dimethacrylate

100 parts sodium chloride solution (0.01 M), tetradecane (2), Blemmer ALE 800 (0.12), triethyleneglycol dimethacrylate (0.12), lauryl peroxide (0.01) and FeSO₄ (0.01) are combined and emulsified under Argon (see above) with an Ultraturrax. After stirring for 18 h, a capsule suspension is obtained.

### Example 7

Oleic Acid Diglyceride-20 EO, Epoxidized and Opened with Acrylic Acid+dodecane-1,12-diol Dimethacrylate

60 parts sodium chloride solution (0.01 M), oleic acid diglyceride-20 EO, epoxidized and opened with acrylic acid (0.9), dodecane-1,12-diol dimethacrylate (0.9), lauryl peroxide (0.07) and FeSO₄ (0.07) are combined and emulsified under Argon with an Ultraturrax (see above). After stirring for 18 h, a capsule suspension is obtained (particle diameter, as measured by light scattering: 200 to 400 nm).

### Example 8

Trem LF 40 (Alkyl Allylsulfosuccinic Acid Sodium Salt, a Product of Cognis Corp.)+Triethyleneglycol Dimethacrylate

60 parts sodium chloride solution (0.01 M), tetradecane (12), Trem LF 40 (0.7), triethylene glycol dimethacrylate (0.7), lauryl peroxide (0.06) and FeSO₄ (0.06) are combined and emulsified under argon with an Ultraturrax (see above). After stirring for 18 h, a capsule suspension is obtained.

### Example 9

Trem LF 40+Triethyleneglycol Dimethacrylate

100 parts sodium chloride solution (0.01 M), tetradecane (2), Trem LF 40 (0.12), triethyleneglycol dimethacrylate (0.12), lauryl peroxide (0.01) and FeSO₄ (0.01) are combined and emulsified under argon with an Ultraturrax (see above). After stirring for 18 h, a capsule suspension is obtained.

### Example 10

Encapsulation of Perfume

100 parts sodium chloride solution (0.01 M), tetradecane (2), 2 drops eucalyptus oil, Blemmer ALE 800 (0.12), triethylene glycol dimethacrylate (0.12), lauryl peroxide (0.01) and FeSO₄ (0.01) are combined and emulsified under argon with an Ultraturrax (see above). After stirring for 18 h, a capsule suspension is obtained. For comparison, an emulsion is prepared without lauryl peroxide or FeSO₄. After 4 days (open), the odor of the capsule suspension is less intensive, but becomes much more intensive on grinding.

### Example 11

Preparation of Diallyl Ammonium Dodecyl Sulfate

144 parts Texapon K 1296 dissolved in water (144) are protonated with conc. hydrochloric acid (49.5) and neutralized with diallylamine (48.5).

### Example 12

Oleic Acid Diglyceride-20 EO, Epoxidized and Opened with Acrylic Acid+Dodecane-1,12-diol Dimethacrylate

439 g triolein (Edenor KL 20) are ethoxylated with 880 g in the presence of 23 g glycerol and 8.8 g 50% potassium hydroxide (autoclave, 180° C., 5 bar). This ethoxylated statistical diglyceride (600 g) is epoxidized for 2 h at room temperature with 224 g m-chloroperbenzoic acid in chloroform in the presence of 120 g sodium hydrogen carbonate. After washing out with a 10% sodium sulfite solution and a saturated sodium carbonate solution and drying, the chloroform is removed in a water jet vacuum at 40° C. A yellow, clear liquid product (251 g) is obtained. 4.9 g acrylic acid, 0.05 g triphenylphosphine and 0.11 g hydroquinone monomethylether are added to 50 g of this epoxidized Edenor KL 20 ethoxylate and the whole is stirred for 6 h.

1. A process for the encapsulation of emulsions, characterized in that a w/o or o/w emulsion is prepared from
(a) a polymerizable emulsifier,
(b) at least one polyfunctional comonomer,
(c) at least one hydrophilic liquid and
(d) at least one hydrophobic liquid,
the resulting mixture is polymerized to obtain a matrix and the hydrophilic and hydrophobic liquids are encapsulated by the matrix.

2. A process as claimed in claim 1, characterized in that a polymerizable emulsifier containing a methacryl, aryl, vinyl or allyl group is used.

3. A process as claimed in claims 1 and/or 2, characterized in that polyfunctional comonomers selected from the group consisting of 1,6-hexanediol diacrylate, 1,12-dodecanediol dimethacrylate, dipropylene glycol diacrylate, triethylene glycol dimethacrylate, trimethylolpropane triacrylate, trimethylolpropane ethoxylate triacrylate, glyceryl propoxylate triacrylate, diallylamine, N,N′-diallyltartaric acid diamide and divinylbenzene are used.

4. A process as claimed in at least one of claims 1 to 3, characterized in that hydrophilic liquids selected from the group consisting of water, glycerol carbonate, polyols, N,N-dimethyl formamide, dimethyl acetamide, dimethyl sulfoxide, N-methylpyrrolidone, glycol ethers, short-chain alcohols, ketones and esters, triacetin and mixtures thereof are used.

5. A process as claimed in at least one of claims 1 to 4, characterized in that hydrophobic liquids selected from the group consisting of aliphatic solvents, aromatic solvents, halogenated aliphatic solvents, halogenated aromatic solvents, fatty alcohols and oil components are used.

6. A process as claimed in at least one of claims 1 to 5, characterized in that the polymerization is initiated by initiators or UV irradiation.

7. A process as claimed in at least one of claims 1 to 6, characterized in that the w/o or o/w emulsion is prepared by addition of active substances as an additional component (f).

8. A process as claimed in at least one of claims 1 to 7, characterized in that capsules with a particle size of 70 nm to 5 μm are obtained.

9. Encapsulated emulsions obtainable by preparing a w/o or o/w emulsion from
(a) a polymerizable emulsifier,
(b) at least one polyfunctional copolymer,
(c) at least one hydrophilic liquid,
(d) at least one hydrophobic liquid,
(e) optionally monofunctional comonomers and
(f) optionally active substances,
polymerizing the resulting mixture to obtain a matrix and encapsulating the hydrophilic and hydrophobic liquids in the matrix.

10. The use of the encapsulated emulsions claimed in claim 9 in surface-active preparations.
